(19) United States
(12) Patent Application Publication
(10) Pub. No.: US 2003/0114069 A1
(43) Pub. Date: Jun. 19, 2003

(54) Personal care and surface cleaning article

(76) Inventors: Gerard Scheubel, Quebec (CA); Albert R. Kelly, New York, NY (US)

Correspondence Address: Evelyn M. Sommer, 30th Floor, 825 Third Avenue, New York, NY, 10022, US

(21) Appl. No.: 10/222,401

(22) Filed: Aug. 16, 2002

Related U.S. Application Data
(63) Continuation-in-part of application No. 10/021,395, filed on Dec. 19, 2001.

(51) Int. Cl. B32B 5/02; B32B 27/04; B32B 27/12; B32B 5/26; B32B 21/02; B32B 21/10; B32B 5/06; B32B 7/08

(52) U.S. Cl. 442/388; 442/381; 442/385; 442/387; 442/389; 442/148

(57) ABSTRACT

Flexible, substantially dry, disposable article suitable as a personal care article for cleaning and conditioning skin, sanitizing and disinfecting hard surfaces, methods of manufacturing and using the same wherein the article comprises: a first water insoluble flexible nonwoven thermoplastic outer layer having an outer and inner surface; a core layer containing a treatment composition adapted for cleaning, sanitizing or disinfecting hard surfaces or for personal cleaning and conditioning and a second water insoluble flexible nonwoven thermoplastic layer having an outer and inner surface positioned so its inner surface is opposite the inner surface of the first layer; the core layer having been mechanically bonded by needling to and between the first and second layers so as to form a unified article having pin dot perforations of an average pore opening size of less than 300 microns and preferably less than 100 microns in diameter formed by the needling extending through all three of said layers. The invention also contemplates such articles which have not been treated with a treatment composition.

This application is a continuation-in-part of application Ser. No. 10/021,395, filed Dec. 19, 2001.

The present invention relates to a substantially dry, flexible disposable article suitable as a personal care article for cleaning and conditioning skin and for cleaning, sanitizing and disinfecting hard surfaces, methods for manufacturing and using the same.

Treated flexible articles for personal care and for cleaning, sanitizing and disinfecting hard surfaces are known. These articles are generally single ply structures that have been coated, sprayed or impregnated with a treatment composition that is activated on being wetted with water. The articles with the treatment composition incorporated therein are substantially dry to the touch. A disadvantage of these articles is when large surfaces are involved or when it is desired that the article be used for more than one use, necessitating the rewetting of the article to reactivate the treatment composition incorporated therein, that on the first wetting of the article, the active ingredients are substantially completely released such that the intended larger or multi stage cleaning task cannot be completed. Another disadvantage of the available cleaning articles is that the use of higher levels of treatment composition adversely affects hand-feel, and the heavily treated articles feel wet, lubricious or boardy.

A key requisite for producing longer-lasting cleaning articles is inclusion of enough treatment composition for the tasks intended, and provisions both for accelerating activation of that composition by water and for controlling the release of the activated treatment composition over time. Traditionally, this had been accomplished with the use of concentrated treatment compositions that contain little or no water, that are solid or semi-solid at room temperatures, that are coated onto the surface of a flexible substrate at elevated temperatures, and that are allowed to dry as a thin coating at ambient temperatures. The disadvantages of these types of coatings include lubricity of the wax-like coatings, boardy hand-feel and an inability to control activation or release; all of which depend upon the chemistry of the treatment compositions.

In an earlier filed application, Ser. No. 10/021,395, a disposable flexible article useful for personal care and cleaning hard surfaces is disclosed comprising a first water insoluble flexible nonwoven thermoplastic layer, a second water insoluble flexible nonwoven thermoplastic layer, an intermediate core layer of a nonwoven material, the core layer being ultrasonically bonded to and between the first and second layers so as to form a unified article having pin dot perforations extending through all three layers.

The applicants have now found that mechanical web bonding or needling can be utilized for bonding the first and second layers with the core layer between the first and second layers to form a uniform article having pin dot perforations extending through all three of the layers.

An object of this invention is to provide a substantially dry, flexible cleaning article useful for cleaning, sanitizing and disinfecting hard surfaces that can be activated with water and reactivated for continued or repeated use until the cleaning task is or tasks are completed.

Another object of this invention is a flexible, substantially dry, cleaning article having incorporated into its structure, treatment composition in an amount of from 25 to 600 percent of the structure's total basis weight without the hand feel being adversely affected and without any loss of treatment composition from the article prior to actual use thereof.

It is another object of the invention to provide such cleaning articles which are disposable without giving rise to ecological or other problems.

Still another object is a cleaning article adapted for controlled and repeated release of the active ingredients present in the treatment composition incorporated in its structure.

Yet another object is to provide a cleaning article adapted for controlled and repeated release of the active ingredients present in the treatment composition incorporated in its structure adapted for use for personal care.

Yet another object is to provide a cleaning article adapted for controlled and repeated release of the active ingredients present in the treatment composition incorporated in its structure adapted for use for cleaning, sanitizing and disinfecting hard surfaces.

Still a further object of the invention is to provide a cleaning article adapted for controlled and repeated release of the active ingredients present in the treatment composition incorporated in its structure having different surface textures on opposite sides thereof.

Yet another object of the invention is to provide a cleaning article adapted for controlled and repeated release of the active ingredients in the treatment composition incorporated in its structure having an outer surface that can be imprinted for labeling, decorative or instructional purposes.

Disposable articles as herein disclosed are intended for single use, as well as multiple uses, i.e., up to 12 and more uses and as such are more disposable than sponges, cloths which can develop bacterial growth, unpleasant odors and other undesirable characteristics.

These and other objects will become readily apparent from a reading of the detailed description of the invention that follows.

The present invention relates to a flexible, substantially dry, disposable article suitable as a personal care article for cleaning and conditioning skin, and for cleaning, sanitizing and disinfecting hard surfaces, said article comprising:

1. a first water insoluble flexible nonwoven outer layer having an outer and an inner surface and comprised substantially of thermoplastic fibers,

2. a core layer formed at least in part of a cellulosic material and

3. a second water insoluble flexible nonwoven layer having an outer and inner surface comprised substantially of thermoplastic fibers positioned so its inner surface is opposite the inner surface of the first layer, the core layer being mechanically bonded to and between the first and second layers by needling so as to form a unified article having small perforations on the order of less than 200 microns formed by said mechanical bonding, i.e., needling extending through all three of said layers.

The invention also contemplates flexible substantially dry disposable articles comprising a first water insoluble flexible nonwoven thermoplastic outer layer having an outer and an inner surface, a core layer formed at least in part of a cellulosic material containing a treatment composition adapted for cleaning, sanitizing or disinfecting hard surfaces or for personal cleaning and conditioning and a second water insoluble flexible nonwoven thermoplastic layer having an outer and inner surface positioned so its inner surface is opposite the inner surface of the first layer, the core layer being mechanically bonded by needling to and between the first and second layers so as to form a unified article having perforations per inch of at less than 300 microns in diameter formed by said mechanical bonding, i.e., needling extending through all three of said layers.

When the term “thermoplastic layers” is used, it is intended to include layers which are entirely or substantially composed of thermoplastic fibers. It is however possible for the thermoplastic layer to include a minor amount of natural fibers such as rayon, cotton, viscose or lyocell.

Cellulosic materials suitable for use in forming the core layer are fibers composed or derived from cellulose. Examples are cotton (cellulose), hemp and paper. Particularly preferred fibers are derived from wood pulp. The cellulosic core used can be an airlaid nonwoven, a spun lace hybrid containing a substantial amount of pulp fibers, a tissue paper, a creped paper, wadding or other form of roll like cellulosic materials.

The term mechanical bonding is taken to mean the bonding together of fiber webs by using needles. The idea of bonding fibrous material, usually composed of coarse natural fibers or recycled cuttings or waste, by vertical perforation by many barbed needles to make a web, is a very old one. The technique has been used in industry since the 1870s. It enables textiles to be made for special purposes, such as upholstering, insulating, and dampening, either out of fibers which were difficult to spin or could not be spun at all from fibers that could not be felted, such as wool, and animal hair.

In 1952 DuPont made the first attempts at needling synthetic fibers having been unsuccessful in its efforts to produce man-made fibers which had felting properties similar to wool. They were trying to replace the felting process that takes place in wool with the needling of carded web made from synthetic fibers and to replace the felting process by shrinking the needled material. High quality synthetic fibers with new properties began to appear and new uses were found for them, which gave the incentive for further development of needling techniques, e.g. for needle punched floor coverings.

The mechanical bonding of fiber webs with needles is a technique now employed in the production of numerous products which not only compete with conventionally made textiles, but exhibit properties hitherto unknown and not attainable by any other method.

The properties of needled fabrics depend mainly on the kind of fiber; the method by which the web is made; the needling technique employed together with the needle tooling. In needling the bonding of the fiber web is the result of intertwining of the fibers and of the increased interfiber friction caused by the compression of the web.

Different types of needles are available and can be used to produce different types of effects, more particularly the size of the needles, the number and shape of the barbs in the working zone, determine the amount of fibers being reoriented due to the action of said needles, and so create the densified structure required. Needling's aim is to create a cohesive structure, densify this web structure and bond fibers together. Needling also has the capability to perforate cohesive structures such as paper like structures or films and transfer fibers from the fibrous structure through the action of the barbs located on each needle through the cohesive structure. By reaching another fiber based web, it creates an intimate bond within all layers. Thus the needle used can determine the width and depth of the openings and the amount of the fibers that has to be reoriented. Needling's aim has been to compress and bond the fibers together.

While needle punching has been available for some time, the technique has been applied to synthetic or natural fibers having sufficient length to be carried by the needles. Single webs are most frequently used and while it is known to use needle punching for mechanically bonding more than one web to form a composite, the fibers from which the webs are formed while differing in properties, i.e., flexural strength, tensile load and extensibility as well as in thickness, length and distribution of strength, have all been synthetic. Needling without encountering considerable difficulties was not believed possible with cellulosic materials and particularly wood pulp derived materials because of the short length of the natural fibers, their fineness etc. The inventors herein have now found that the composite of the invention, inner and outer nonwoven thermoplastics enclosing there between a cellulosic layer or a layer in the main formed of cellulosic fibers, can be mechanically bonded together by needle punching. The needling joins together the different fiber webs or layers by piercing them evenly with the needle to push small punches of fiber there through, the nature of the cellulosic web having been predetermined to insure that it will maintain its integrity during the needling process.

The conventional needling machines are used. Suitable machinery and processes are described on Non-Woven Bonded Fabrics, Editors, J. Lunenschloss and W. Albrecht, Ellis Horwood Series in Industrial Technology, Ellis Horwood Limited, Chichester.

In another aspect of the invention, additional layers of thermoplastic materials such as dry laid resin bonded fabrics, heat bonded fabrics, spun bonded or hydro entangled fabrics based on polypropylene and/or polyester or needle punched polypropylene or polyester may be disposed adjacent to the outer layers or used to form the bottom surface or the top surface, of the cleansing article respectively so that articles of 4, 5, 6 or seven layers are possible, however in all instance the innermost three layers are those described herein.

Preferably, the first and second outer layers are composed essentially of thermoplastic fiber based nonwovens selected from polyesters, polyolefins, polyamides and most preferably spun laced, hydroentangled polyester, or polypropylene and needle punched polyester or polypropylene. The fibers can be made of single polymer component or can be bicomponent fibers having a side by side or sheath core structure. The first and second outer layers or additional layers provided on the first and second layers can also be comprised of a mixture of the thermoplastic fibers having abrasive particles incorporated therein or which can be flame treated or singed to form roughened or hardened surfaces. The abrasive particles are added in order to form a rough or abrasive surface preferably on at least one face of the article. Abrasive particles can be created by melting locally thermoplastic polymers thus creating polymer hard points through partial fusion of fiber ends or fiber bundles. The heat treatment applied can be a local intense heating provided by an open flame in a process called singeing or high intensity infrared heating or similar methods capable of melting only parts of the fibrous structure. Another aspect of this invention is to apply resin finishing treatments on the outer surface of the desired layer taking care of hardening only locally said layer. In accordance with another embodiment of this invention a supple fabric is provided containing metallic fibers of appropriate size blended to the thermoplastic fibers in order to impart roughness to the desired layer. The metallic fibers can be selected amongst stainless steel fibers or any other metallic fiber such as soft iron. An outer layer can also be embossed or debossed ultrasonically so that it exhibits a pattern of raised and depressed areas or surface aberrations as an alternative to including abrasive particles or flame treating and the like. This abrasive or embossed surface also serves to enhance the cleaning or exfoliating effect in the case of the personal care article and the cleaning and scouring effect in the case of the article for cleaning hard surfaces.

The thermoplastics nonwovens can incorporate coloring substances therein so that they present a varied appearance for example with the outer layers being of the same or different colors.

To form the disposable cleaning article of this invention, a core layer is mechanically through-bonded by needling while it is positioned between the outer layers using needling machinery using conditions sufficient to achieve the bonding of the elements and to perforate all of the layers effectively. The perforations of the layers have a diameter of less than 300 microns and preferably less than 100 microns and are rendered fluid permeable. The perforations help control and meter water absorption, activation and release of the cleansing or treatment component. If more than three layers are present, the bonding and formation of apertures or perforations is achieved in the same manner and to the same effect.

The term “disposable” as used herein means that the needle bonded and through perforated articles are designed to be reactivated a limited number of times and then discarded, preferably less than about 12 to 24 times and most preferably more than a single usage event. Through-bonding while causing perforations has not been practiced or commercialized heretofore where webs of natural material such as cellulose, wood pulp, hemp, cotton, jute and the like were involved in the manufacture of the article. For the purpose of the instant invention articles, all of which are specifically designed for disposability, through-bonding enhances performance and mandates disposability by facilitating controlled de-lamination to be engineered into the product's design specifications.

The term “substantially dry”, as used herein, means that the articles exhibit a hand feel that may feel slightly lubricious, but not water-wet.

The disposable cleaning articles of the invention must comprise a water insoluble inner core element and at least two outer water insoluble surface contacting elements with at least one outer surface element on each side of the core element, wherein the article contains apertures of less than 300 microns and preferably less than 100 microns in diameter extending through all three of the elements or layers. The core element may include a cellulosic (paper) or other nonwoven web, i.e., a web composed in the main of cellulosic fibers having absorbent capacity, compressibility, controlled retention and dispensing rates and wet strength for use and physical strength. In its preferred embodiment, the core element should have an absorbent capacity of up to twenty grams per gram of core material. The wet wicking rate of the core should be less than about 25 seconds. The base weight of the core should be from about 15 to about 100 grams per square meter. Materials for the core are selected from the group of cellulosic, natural materials such as cotton, jute, hemp, wood pulp, and mixtures thereof, which should preferably be of low density, not exceeding 0.5 grams per cubic centimeter.

The cellulosic core is important as is the needle punching since there results a composite effective to

1. inhibit migration of the treatment composition incorporated therein,

2. reduce the loft, folded wiper stack height,

3. assist in the metered release of the incorporated active ingredients upon activation with water,

4. expand and soften the hand of the activated wipes.

After formation of the composite article, the article can be treated in the conventional manner by impregnation, dipping, spraying or coating the article with a treatment composition, the treated article remaining substantially dry to the touch. The article may also be treated so that it includes the treatment composition comprising the active ingredients in the form of a substantially dry composition that does not permeate or impregnate either of the outer layers, and effectively resides between the inner surfaces of the first and second or outer layers.

The first and second and any additional outer layers and the core are bonded together to maintain the integrity of the article and to provide the article in the thermoplastic outer layers with apertures such that the composite layers become fluid permeable. The apertures encourage the introduction of water or other fluids into the core to activate the treatment composition that has been impregnated, coated, sprayed or otherwise incorporated therein. The application of water, pressure and friction facilitates and controls the metered release of the activated composition.

The bonding of the core with the outer layers is executed mechanically by needling and has to be conducted so all of the layers are completely perforated. The needling is conducted in the conventional manner, utilizing the conventional needling equipment and needles adapted for producing the through perforations as described herein.

The invention includes the novel needle punch composites per se as well as articles containing a treatment composition.

The cleaning articles of the invention contain in the core layer a treatment composition which has been added onto or impregnated and penetrates into the core layer and is releasably associated therewith.

The treatment composition for the article to be used for personal cleansing can include, in addition to surfactants and preferably lathering surfactants, emollients, lubricants, protectants, deodorants or medicaments.

The apertures should preferably be uniform in size and shape and of small size, preferably having an average diameter of less than 300 microns and preferably less than 100 microns. In addition to the two layers and core, other insoluble layers may be present disposed adjacent to the outer surface(s) of the two layers. These additional layers can include abrasives and other non-fibrous materials. The bonding of the multi layers takes place mechanically by needling and is regulated so as to bond the layers and to produce apertures or openings extended into and through all of the layers.

The articles intended for cleaning hard surfaces can have incorporated therein compositions as described in U.S. Pat. No. 6,141,644 the entirety of which is incorporated herein by reference.

The treatment composition for the article when it is to be used for cleaning, sanitizing and disinfecting hard surfaces, can include, in addition to surfactants, an antiseptic, antibacterial, wax, waterproofing, polishing or other agents as are conventionally used in cleaning compositions intended for cleaning and protecting hard surfaces in homes, restaurants, hospitals, nursing homes for private and industrial use and the like. They can be used as mopping and dusting cloths, and as polishing and cleaning cloths.

In a preferred embodiment for personal skin care, the treatment composition includes a surfactant and at least one member of the group of emollients, lubricants, conditioning agents, protectants, deodorants and medicaments. When the article is exposed to water at the point of use and pressure applied, as by squeezing, an unstable emulsion and in the case of the use of a foaming surfactant, a lather is formed which releases the components of the cleansing treatment composition onto the skin or hair of the individual. The treatment compositions used in connection with personal skin care articles is most advantageously used to provide 25 to about 600 percent of treatment composition referred to the final articles total basis weight.

The outer layers of the article of the invention may have different textures and abrasiveness. Differently textured surfaces can be adapted so as to provide an abrasive side for more intensive cleaning and a softer absorbent side for lighter and more gentle cleaning.

The surfactants employed in the treatment compositions are preferably lathering surfactants, but this is not required and may include anionic, cationic, amphoteric and non-ionic surfactants either separately or in combination and preferably include as anionic surfactants, the phosphates, taurates, sulfates, sarcosinates, isothionates, etc, for example ammonium lauroyl sulfate, sodium lauroyl sarcosinate, sodium lauroyl lactylate, etc.

Nonionic surfactants which can be used include amine oxides, alkoxylated fatty acid esters, polyhydroxy fatty acid amides, alkyl glycosides and the like exemplified by lauramine oxide, sucrose laurate, sucrose cocoate and the like. Instances of suitable amphoteric surfactants are the amino alkanoates, alkyl amino acetates, hydroxy, sultaines, betaines, etc., as exemplified by sodium-3-dodecylaminopropinate, disodium lauroamphodiactetate and sodium lauroampho acetate.

The substantially dry core is interposed between the two water insoluble outer layers, and the resultant lamination is mechanically bonded to form a perforated uniform article and is then treated with the treatment composition.

Preferably the article of the invention comprises three discrete elements that have been mechanically through-bonded by needling to form a uniform perforated article. For the purpose of the mechanical bonding, the outer layers are preferably composed of similar synthetic fiber materials and the inner layer or core layer of a cellulose material and preferably a wood pulp derived material such as paper.

The disposable articles of the invention can accommodate from 25 to about 600 percent of treatment composition of the resulting structures' total basis weight without any loss of their excellent hand properties including feel, drape, conformability and flexibility.

The disposable articles of the invention are substantially dry and are intended to be activated by water prior to use by immersion in water, by placing it under a stream of water or by placing it in contact with a wet hard surface or wet skin. The treatment composition is released in active form by squeezing or rubbing the article prior to or during contact of the article with the surface to be treated or cleaned.

The articles all have in common a high water absorption capacity achieved by using the absorbent cellulose fiber core. The apertures formed in the needling guarantee the unhindered passage of water into and treatment composition out of the device, the apertures being capable of modification to influence how the water gets in the article and amounts of the composition out of the article.

In order to describe the invention more fully and not by way of limitation, the following examples are presented.

1. THREE-PLY HARD SURFACE CLEANING CLOTHS—A single inner absorbent core element comprising a 28# cellulosic material having a density of about 0.1 gram per cubic centimeter is interposed between a web of 1,2 ounce per square yard polyester material made of 3 denier fibers and 2.3 ounce polypropylene fiber material made of a blend of coarse fibers of 17 and 60 denier. The three elements are bonded mechanically together by needling to form a flexible article of controlled density. A calendar compression treatment is applied between two metallic rolls in order to achieve a thickness of 1/20^{th} of an inch and a pore opening of less than 100 microns. The resulting cloth when wet with water, and wrung dry provided an excellent cleaning. The cloth exhibited a high water absorption capacity. It could be wet and wrung out many times without its absorbency being affected. When wrung out it could also be used to dry hard surfaces that had been wetted.

2. THREE-PLY HARD SURFACE CLEANING CLOTHS—A single inner absorbent core element comprising a 28# cellulosic material having a density of about 0.1 gram per cubic centimeter is interposed between a 1.2 ounce per square yard of hydroentangled polyester fabric made of 1.7 denier fibers and a 2.3 ounce polypropylene fiber material made of blend of coarse fibers of 17 and 60 denier. The three elements are bonded mechanically together by needling to form a flexible article of controlled density. A calendar compression treatment is applied between two metallic rolls in order to achieve a thickness of 1/20^{th} of an inch and an average pore opening size of less than 100 microns. Because the treatment composition is impregnated under pressure, and the treated composite is passed through a series of nip rolls and folding ploughs, over 80% of the treatment is absorbed into the cellulosic core. This treated core element weighs approximately 126 grams per square yard. The resulting cloth is then placed under running water and squeezed several times to provide a rich slowly released lathering foam providing excellent cleaning properties. The process was repeated approximately twelve times and still provided a rich lathering foam. This process was repeated approximately twelve times, at which point the lathering foam diminished significantly, and the article exhibited obvious signs of de-lamination.

3. FOUR PLY HARD SURFACE CLEANING CLOTHS—A single, inner absorbent core element comprising a 28# cellulosic material, having a density of about 0.1 gram per cubic centimeter is imposed between a 1.2 ounce per square yard carded and cross-lapped polyester fabric made of 3 denier fibers and a 2.3 ounce polypropylene fiber material made of 17 deniers. The three elements are bonded mechanically together by needling to form a flexible article of controlled density. A calendar compression treatment is applied to the laminate between two metallic rolls in order to achieve a thickness of 1/22th of an inch and an average pore opening size of less than 100 microns. The compressed fabric is subjected to an open flame treatment in order to impart the abrasion properties to the composite material. The article was impregnated with 90 grams per square yard of a highly concentrated hard surface surfactant containing treatment composition. This treated core element (weighs approximately 126 grams per square yard) and contains the activated treatment composition for ready release. The perforated polyethylene film layer is intended to present a partial fluid barrier that restricts the flow of water in and the outward release of activated treatment. The treated cloth is then saturated by immersion in water and squeezed several times to produce a rich lathering foam. This process was repeated over 24 times before the foaming action began to dissipate, and the article showed visible signs of de-lamination.

4. FOUR PLY HARD SURFACE CLEANING CLOTHS—A single, inner absorbent core element comprising a 28# cellulosic material, with a density of less than 0.1 gram per cubic centimeter is interposed between a 1.3 ounce polypropylene material which has been interfaced with a 0.5 milligram polyethylene film and a 1.0 ounce spun-bonded polyester material. The four layers are then bonded together mechanically by needling so as to form a flexible entity having perforated bonding points of less than 200 microns in diameter arranged in a uniform pin-dot pattern through which fluids can pass into the treated core and the activated treatment following its delivery can be released outwardly. The perforated polyethylene film layer is intended to present a partial fluid barrier that restricts in use the flow of water in and the subsequent release of activated treatment composition. The article is coated in three-quarter inch wide stripes with 60 grams per square yard of a highly concentrated hard surface surfactant containing composition, separated by one-half inch stripes having no coating. The treated cloth was then saturated with water and squeezed several times to produce a rich lathering foam. This process was repeated over 24 times before the foaming action began to dissipate, and the article is easily delaminated.

5. THREE-PLY SKIN CLEANSING CLOTHS—A single, inner absorbent core element comprising 1.3 ounce cellulose (wood pulp derived) fiber weighing approximately 195 grams per square yard is interposed between a 1.3 ounce resin-bonded, polyester material and a 1.0 ounce spun-bonded polyester material. The three elements are then bonded together by needling so as to form a flexible entity having perforated through-bonding points with a diameter of less than 150 Microns arranged in a uniform pin-dot pattern that are visible on the spun-bonded polyester side only. The cloth is impregnated with 124 grams per square yard of a concentrated skin cleansing and conditioning composition. The treated cloth is then saturated under running water and squeezed several times to provide a rich, lathering foam. This process was repeated approximately 12 times, at which point the lathering foam diminished significantly, and the article exhibited some visible delamination.

What is claimed is:

1. A disposable flexible article useful for cleaning, sanitizing and disinfecting hard surfaces comprising:
a) a first water insoluble non woven layer comprised substantially of synthetic fibers having outer and inner surfaces,
b) a water insoluble nonwoven cellulosic core layer, and
c) a second water insoluble non woven layer having outer and inner surfaces wherein the core layer is positioned between the inner surfaces of said first and second layers and the first, second and intermediate core layer are mechanically through bonded by needling to form a unified article having perforations of less than 300 microns in diameter extending through all of said layers, said perforations having been formed during said mechanical bonding.

2. A disposable flexible article useful for cleaning, sanitizing and disinfecting hard surfaces comprising:
a) a first water insoluble non woven layer comprised substantially of synthetic fibers having outer and inner surfaces,
b) a water insoluble nonwoven cellulosic core layer containing a treatment composition adapted for cleaning hard surfaces
c) a second water insoluble non woven layer having outer and inner surfaces wherein the core layer is positioned between the inner surfaces of said first and second layers and the first, second and intermediate core layer are mechanically through bonded by needling to form a unified article having perforations of less than 300 microns in diameter extending through all of said layers, said perforations having been formed during said mechanical bonding.

3. A disposable flexible article according to claim 2 wherein said treatment composition contains surfactant selected from the group consisting of anionic, nonionic, amphoteric surfactants and mixtures thereof.

4. A disposable flexible article according to claim 3 wherein said anionic surfactant is a member selected from the group consisting of sarcosinates, sulfates, isothionates, phosphates, taurates, lactylates, glutamates, and mixtures thereof.

5. A disposable flexible article according to claim 3 wherein said non-ionic surfactant is a member selected from the group consisting of amine oxides, alkyl glucosides, alkyl polyglucosides, polyhydroxy fatty acid amides, alkoxylated fatty acid esters, sucrose esters, and mixtures thereof.

6. A disposable flexible article according to claim 3 wherein said amphoteric surfactant is a member selected from the group consisting of betaines, sultaines, hydroxysultaines, alkyliminoacetates, iminodialkanoates, aminoalkanoates, and mixtures thereof.

7. A disposable flexible article according to claim 2 wherein said article is substantially dry prior to use.

8. A disposable flexible article according to claim 2 wherein at least one additional layer is present having been applied to the outer surface of at least one of said first and second layers prior to the mechanical bonding thereof.

9. A disposable flexible article according to claim 2 wherein at least one of said first and second layers contains an abrasive material.

10. A disposable flexible article according to claim 8 wherein said additional layer has a different texture than said first or second layer.

11. A disposable flexible article according to claim 10 wherein said outermost layers have imparted thereto a different texture formed by singeing or flame treatment of at least one of said layers.

12. A disposable flexible article according to claim 8 wherein said additional layer serves a partial fluid barrier.

13. A disposable flexible article according to claim 10 wherein said different texture has been formed by flame treatment.

14. A disposable flexible article according to claim 2 wherein said treatment composition is present in an amount of from 25 to 300% of the article's total basis weight.

15. A method of manufacturing a disposable flexible article according to claim 1 comprising the steps of placing the core layer between said first and second layers, mechanically through bonding by needling said layers to simultaneously form apertures having a diameter of less than 300 microns throughout said article.

16. A method of cleaning a hard surface with an article according to claim 1 comprising the steps of wetting said article with water and contacting the surface to be cleaned with said article.

17. A method of manufacturing a disposable flexible article according to claim 15 comprising the step of adding a treatment composition to said article.
